(19) United States
(12) Patent Application Publication
(10) Pub. No.: US 2014/0370566 A1
(43) Pub. Date: Dec. 18, 2014

(54) HIGH-NITROGEN LOADING FOR AMMONIA PROCESSING VIA ANAEROBIC DIGESTION

(71) Applicant: R-Cubed Energy, El Paso, TX (US)

(72) Inventors: Keith Hughes, El Paso, TX (US); Mark Simon, Wilbraham, MA (US)

(21) Appl. No.: 14/472,946

(22) Filed: Aug. 29, 2014

Related U.S. Application Data
(63) Continuation-in-part of application No. 13/739,855, filed on Jan. 11, 2013.
(60) Provisional application No. 61/585,476, filed on Jan. 11, 2012.

(51) Int. Cl. C12P 3/00; C12M 1/107

(52) U.S. Cl. CPC C12P 3/00; C12M 21/04
USPC 435/168; 435/290.4; 435/290.2

(57) ABSTRACT

A method and system to improve in vitro anaerobic digestion processes are disclosed. Simultaneous digestion of dairy manures with various food wastes improves anaerobic process stability and methane production. Co-digestion with blood meal and sweet clover (“BMSC”) at the proper concentrations improves nutrient balance and digestion, biogas production, gives more predictable ammonia concentrations, enhances nutrient content of soil amendment products, and increases the potential for production of ammonia-based fertilizer synthesis. Balanced introduction of BMSC with dairy manure increases methane production, reduces or eliminates co-digestion process limitations, and simplifies storage and delivery of the co-substrate. Following digestion, downstream or back-end products can be produced, including methane, and ammonium based fertilizers. Embodiments advantageously provide a treatment methodology for increased methane production while minimizing the anaerobic digestion process limitations from the use of raw animal wastes exclusively.

### CROSS REFERENCE TO RELATED APPLICATIONS

This patent application is Continuation-in-Part of U.S. Non-Provisional patent application Ser. No. 13/739,855 filed on Jan. 11, 2013 and entitled “HIGH-NITROGEN LOADING FOR AMMONIA PROCESSING VIA ANAEROBIC DIGESTION,” which is hereby incorporated by reference in its entirety. This patent application claims the benefit under 35 U.S.C. §120 of the preceding application. U.S. Non-Provisional patent application Ser. No. 13/739,855 claims the benefit under 35 U.S.C. §119(e) of U.S. Provisional Patent Application Serial. No. 61/585,476 filed on Jan. 11, 2012 and entitled “HIGH-NITROGEN LOADING FOR AMMONIA PROCESSING VIA ANAEROBIC DIGESTION,” which is hereby incorporated by reference in its entirety.

### FIELD OF THE INVENTION

The disclosed embodiments relate to in vitro anaerobic digestion through the use of an anaerobic digester device. The disclosed embodiments further relate to biogas production. The disclosed embodiments also relate to co-digestion to improve yields of biogas.

### BACKGROUND

Anaerobic digestion devices further degrade organic materials using microbial organisms under controlled anoxic conditions. The disclosed embodiments have been designed to be used with all anaerobic digesters such as covered lagoon, plug flow and complete mix digestion devices as examples. The combined benefits for waste management and potential revenue generation from electrical energy, fertilizer, and other products have promoted the growing use of anaerobic digestion technologies, particularly in animal processing and agriculture. As one of the most efficient waste and wastewater treatment technologies, anaerobic digestion is also widely used for the treatment of organic industrial wastes. Digestion produces microbial biomass and biogas, a mixture of carbon dioxide and methane, a renewable energy source.

The anaerobic digestion process begins with bacterial hydrolysis of insoluble organic polymers such as carbohydrates and proteins, converts them to amino acids and sugars. Acidogenic bacteria then convert the sugars and amino acids into carbon dioxide, hydrogen, ammonia, and organic acids. Acetogenic bacteria convert the resulting organic acids into acetic acid, along with ammonia, hydrogen, and carbon dioxide. The methanogen bacterial group then converts these products to methane and carbon dioxide.

The anaerobic digestion process is slowed or halted by inhibitory materials created during the process. A material is inhibitory when it causes an adverse shift in the microbial population or an inhibition of bacterial growth during digestion. A high ratio of inhibitory products in a digestion substrate mixture can accumulate, along with process-inhibiting fatty acids. Methanogen growth and thus methane production is thus inhibited.

Accordingly, there exists a need for an improved anaerobic digestion process utilizing co-digestion.

### SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is therefore an object of the disclosed embodiments to provide for improved anaerobic digestion through the use of an anaerobic digester device by means of nutrient enhancement.

It is another object of the disclosed embodiments to provide for improved biogas production.

It is an additional object of the disclosed embodiments to provide an improved co-digestion process for improved yields of biogas.

The above and other aspects can be achieved as is now described. A method and system to improve anaerobic digestion are disclosed. Simultaneous digestion of dairy manures with various high protein containing food wastes improves anaerobic process stability and methane production. Co-digestion with blood meal and sweet clover (“BMSC”) at the proper concentrations improves nutrient balance and digestion, biogas production, gives more predictable ammonia concentrations, enhances nutrient content of soil amendment products (e.g., digester bottoms), and increases the potential for ammonia-based fertilizer synthesis. Balanced introduction of BMSC with dairy manure increases methane production, reduces or eliminates co-digestion process limitations, and simplifies storage and delivery of the co-substrate. Following digestion, downstream or back-end products can be produced, including methane, digester bottoms, and ammonium based fertilizers. Embodiments advantageously provide a treatment methodology for increased methane production while minimizing the anaerobic digestion process limitations from the use of raw animal manures and wastes.

### BRIEF DESCRIPTION OF THE FIGURES

The accompanying figures, in which like reference numerals refer to identical or functionally-similar elements throughout the separate views and which are incorporated in and form a part of the specification, further illustrate the embodiments and, together with the detailed description, serve to explain the embodiments disclosed herein.

FIG. 1 illustrates an exemplary pictorial illustration of a single stage, standard rate, anaerobic digester device, in accordance with the disclosed embodiments;

FIG. 2 illustrates an exemplary pictorial illustration of a multi stage, high rate, anaerobic digester device, in accordance with the disclosed embodiments; and

FIG. 3 illustrates an exemplary high level flow chart of a method and system for high-nitrogen loading for ammonia processing via anaerobic digestion, in accordance with the disclosed embodiments.

### DETAILED DESCRIPTION

The particular values and configurations discussed in these non-limiting examples can be varied and are cited merely to illustrate at least one embodiment and are not intended to limit the scope thereof.

The embodiments will now be described more fully hereinafter with reference to the accompanying drawings, in which illustrative embodiments of the invention are shown. The embodiments disclosed herein can be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. As used herein, the term “and/or” includes any and all combinations of one or more of the associated listed items.

The terminology used herein is for the purpose of describing particular embodiments only and is not intended to be limiting of the invention. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Anaerobic digestion (“AD”) is the conversion of organic material such as slurries, energy crops, and food waste by micro-organisms in a sealed airtight environment. An anaerobic digester device (ADD) is an air tight, oxygen-free vessel that is fed an organic material, such as animal manure or food scraps. Anaerobic biological processes occur within the ADD which produces methane gas, commonly known as biogas, along with an odor-reduced, nutrient-rich effluent. An ADD is therefore designed to encourage the growth of anaerobic bacteria, particularly the methane producing bacteria (the methanogens) that decrease organic solids by reducing them to soluble substances and gases, primarily carbon dioxide and methane.

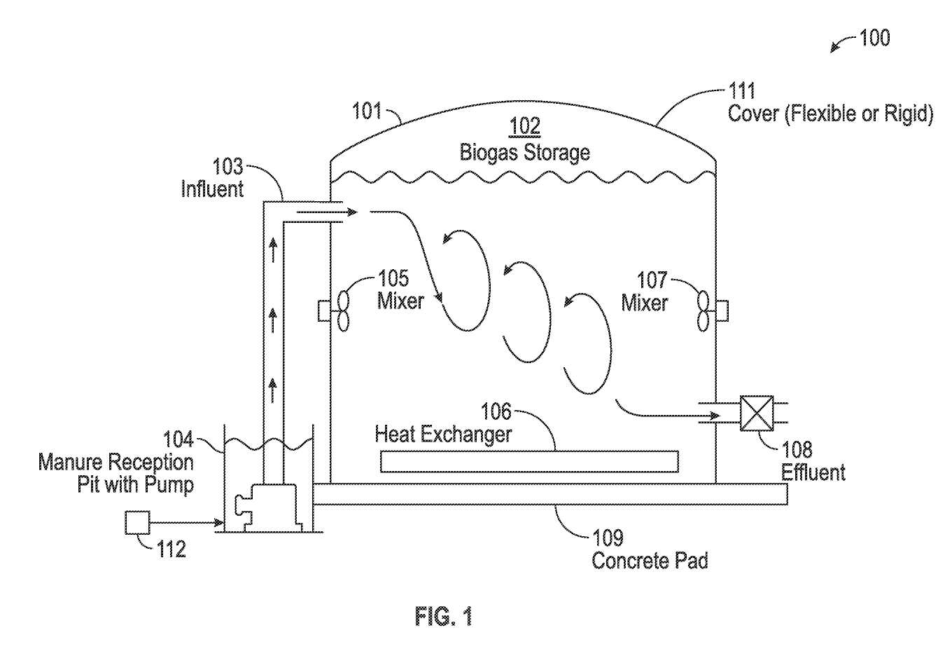

FIG. 1 illustrates an exemplary pictorial illustration 100 of a single stage, standard rate, anaerobic digester device, in accordance with the disclosed embodiments. Organic waste is retained in the ADD for 20 to 40 days and is fed continuously or in batches. The temperature, mixing, and pH are monitored and precisely controlled in the ADD. Completely mixed ADD systems consist of a large tank or vessel 101 where fresh material is mixed with partially digested material. These systems are suitable for manure or other agricultural-food inputs with lower dry matter content (4%-12%). Material with higher dry matter content will work in completely mixed systems by recirculating the liquid effluent. The ADD is a heated tank/vessel 101 with a mechanical, hydraulic, or gas mixing system. Instrumentation is used to closely monitor and adjust temperature, pH, ammonia, and other chemical parameters. A hot water, steam or thermal fluid boiler supplies the heat for the ADD. Control of pH and nutrient supplementation (i.e., introduction of blood meal and sweet clover) is achieved using industrial pumping systems.

The single stage digester in FIG. 1 comprises a vessel 101 with an attached manure reception pit with a pump 104 for pumping influent 103 into the vessel 101. Blood meal and clover additives 112 enter the vessel through the pump in the manure reception pit 104. Once influent 103 enters the vessel 101, mixers 105 and 107 mix the influent with the clover and blood meal 112 for efficient digestion. Biogas storage 102 is located at the top of the vessel 101. A heat exchanger 106 is used inside the vessel to heat the influent 103 and added clover and blood meal 112. A flexible or rigid cover 111 covers the vessel. When completed, effluent 108 exits the vessel 101.

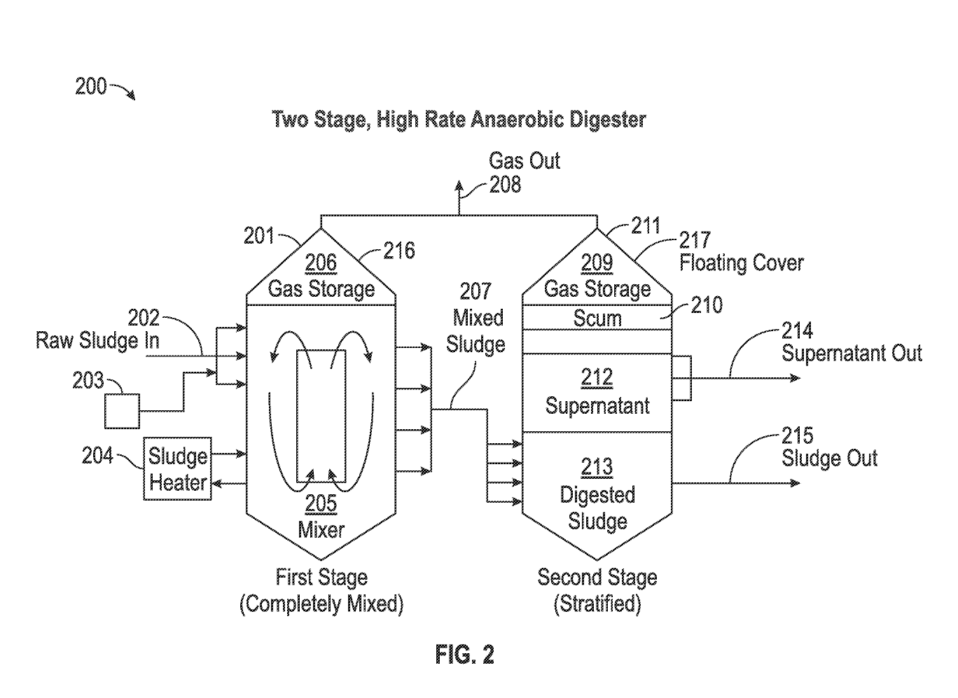

Single stage digester design uses one vessel or container for processing organic material; multi-stage digesters have more than one vessel in series. FIG. 2 illustrates an exemplary pictorial illustration 200 of a multi-stage, high rate, anaerobic digester device, in accordance with the disclosed embodiments. Multiple stage digester designs are optimized at each stage to support maximum methanogen activity and therefore produce more methane. Multi-stage reactors are generally split into 2 stages in two tanks 201, 211, with hydrolysis taking place in the first vessel 201 and the methanogenesis taking place in the second vessel 211. Multi-stage digester design affords better control of digestion kinetics and thus increases the biogas yield.

Both single and multiple stage digesters operate using the same fundamental principles. Waste is collected in a reception pit or tank and pumped 202 into the main reactor vessel(s) 201. Clover and blood meal 203 are also pumped into the first vessel 201. Water is added to achieve between 12 and 15% dry solids. The waste is macerated to reduce particle size to 12 mm if necessary. The reactor vessel(s) 201 contain a stirring or agitation system 205. Biogas storage 206 is located at the top of the vessel 201. Heat from a sludge heater 204 is continuously applied for a 20-40 day incubation period. The biogas is extracted 208 from the biogas storage 206, 209 and treated to remove hydrogen sulfide and water vapor and can then be used by a combined heat and power internal combustion engine to produce electricity.

The mixed sludge 207 is then pumped to the second vessel 211 where methanogenesis takes place. In the second vessel 211, the supernatant 212 is separated from the digested sludge 213 and scum 210. The supernatant exits 214 the second vessel from a separate location than the exit 215 for the digested sludge. Biogas is extracted from the biogas storage 209 from the second vessel 211. Covers 216, 217, either solid or floating, cover the vessels 201, 211.

ADD devices can be operated at lower temperatures (Mesophilic) or higher temperatures (Thermophilic). Mesophilic digesters operate in the 30-38° C. range and thermophilic digesters operate from 49-57° C. Different types of bacteria survive in different temperature ranges. Digester temperature is related to the time and space required for digestion and the required level of sterilization of the digestate. Mesophilic systems need a longer treatment time (retention times of at least 15-25 days or more) in order for the lower temperature micro-organisms to break down organic matter. Thermophilic processes require less retention time and produce more methane gas, but are extremely prone to upset conditions. Thermophilic processes are less robust and require a higher degree of maintenance and have higher operational costs. In general, mesophilic systems are reported to be more robust when considering possible temperature upsets. Small and mid-sized agricultural-food systems will typically operate in the mesophilic temperature range. Some systems are specifically designed to concentrate the solids content to reduce the average overall retention time needed in a mesophilic system.

Anaerobic digestion, through the use of anaerobic digestion devices, traditionally utilizes a single substrate (such as animal manure or municipal sludge). Biogas production can be limited to the nutrient and fat content of the digestion medium. Co-digestion is defined as the simultaneous digestion of a homogenous mixture of two or more substrates. The most common co-digestion method occurs when a major amount of the main basic or primary substrate (e.g. manure or sewage sludge) is mixed and digested together with minor amounts of a single, or a variety of additional co-substrates of higher nutrient content. Simultaneous digestion of dairy manures with various food wastes increases anaerobic process stability. Co-digestion improves nutrient balance and digestion, biogas production, gives more predictable ammonia concentrations, enhances nutrient content of soil amendment products, and increases the potential for ammonia-based fertilizer synthesis.

Utilizing co-substrates in anaerobic digestion improves the biogas yield due to the positive synergisms established in the digestion medium. In digestion, high fermentation rates from proteins and fats from animal derived co-substrates result in the formation of inhibiting substances (e.g., ammonia, sulfides, volatile fatty acids (“VFA”)). Inhibiting substances retard growth of methanogen species and thus reduce methane production. Co-substrates, such as blood meal and sweet clover (“BMSC”), have lower fat content and provide nutrients missing from digestion that prevent inhibiting substances from affecting methanogenesis. Balanced introduction of BMSC with dairy manure increases methane production, reduces or eliminates co-digestion process limitations, and simplifies storage and delivery of the co-substrate. Following digestion, downstream or back-end products can be produced, including methane, soil modifiers and ammonium based fertilizers.

The introduction of a co-substrate improves digester stability and results in the generation of more methane. The use of high protein and high fat animal wastes (e.g., slaughterhouse wastes) typically yields the highest methane production when compared to the digestion of only the main substrate (e.g., manure). Slaughterhouse waste is prone to decomposition with subsequent reduction of nutrient content during both storage and transportation. Process stability of co-digestion, however, using animal wastes (e.g., blood and tissue) is dependent on digester operations that allow the microbiological consortia to adapt to the co-substrate. During digestion, the decomposition of protein results in high concentrations of toxic ammonia gas and less toxic ammonium salts.

High fermentation rates of proteins and fats during digestion result in the formation of inhibiting substances (e.g., ammonia, sulfide, VFA) that retard growth of methanogen species and thus reduce methane production. Elevated lipid concentrations impact plant maintenance and can adversely affect digester performance via washout. Proper handling, pumping, and sanitary storage of the animal wastes must be considered in the overall plant design. European environmental standards require pasteurization of slaughterhouse and raw animal wastes before being used as a digestion co-substrate. Pasteurization eliminates causative agents associated with bovine spongiform encephalopathy (e.g., Mad Cow disease).

Free ammonia is inhibitory to the digestion process and is toxic to the acetate-utilizing methanogens that are responsible for creating 70-80% of the methane produced. The release of ammonia, during protein break-down, gradually increases the process pH. A rise in pH value to 8 units or greater becomes growth limiting for many of the VFA consuming methanogens. The higher than optimal pH, together with a high fermentation rate of proteins and fats in slaughterhouse wastes, can lead to the accumulation of volatile fatty acids. Thus, if the organic load is not decreased at that point, the overload can lead to increasing concentrations of process inhibiting VFA and finally to a total inhibition of methanogenesis with inevitable process collapse.

Maintaining high levels of ammonia in the digestate liquid phase is highly desirable from a co-product manufacturing and economic perspective. In most cases, the lower ammonia concentrations generated from the exclusive digestion of dairy manure does not economically allow for the capital expense of an ammonia recovery system. Ammonium nitrate, sulfate, and citrate have good market values therefore the subsequent marketing and sale of these products can create profit margin and accelerate return on investment.

The sulfur found in animal tissue is also a major contributor to the formation of inhibiting sulfides during anaerobic digestion. The presence of ions such as sodium, calcium, and magnesium supplied by the co-substrate has also been found to be antagonistic to digester inhibition. Antagonism is a phenomenon in which the toxicity of one ion or molecule is decreased by the presence of other ions or molecules. Increasing concentrations of sulfide lead to higher concentrations of hydrogen sulfide in the biogas. High concentrations of hydrogen sulfide can also trigger sulfide inhibition of methanogens.

Co-digestion with blood meal and sweet clover (“BMSC”) at the proper concentrations increases amounts of digestible protein and nitrogen, while limiting lipid content of the feed stock. Lipids (i.e., fats) from animal tissues can attach to the digester media and cause coagulation, flotation, and eventual wash-out of zoological mass. Lipids can accumulate in process critical plumbing and place additional burden on plant maintenance and performance. Increasing protein content of the feed stock results in higher methane concentrations with a 30-40% increase in methane production possible.

Embodiments disclosed herein provide for two exemplary high protein, low fat co-substrates, blood meal and sweet clover, utilized as co-digestion substrates/additives to improve methane production from the anaerobic digestion of dairy manure. Embodiments advantageously provide a treatment methodology for increased methane production while minimizing the outlined process limitations from the use of raw animal wastes. Granular or pelletized blood meal and dried sweet clover can be easily shipped, transferred, and stored without the necessity for stringent health and safety procedures normally associated with using slaughter house wastes as co-substrates.

Co-digestion improves the carbon to nitrogen nutrient balance (C/N ratio) which results in improved digester performance and greater biogas yields. Co-digestion of dairy manure with solid slaughterhouse waste gave biogas yields of 0.8 to 1 m³/kg VS (cubic meters of biogas per kilogram of volatile solids digested).

Organic and/or inorganic supplements with high nitrogen contents can be loaded into the digesters along with the biological materials to create a high-nitrogen effluent. The nitrogen in the effluent is, after the mesophilic digester process, predominantly in the form of ammonia. An exemplary substrate and additive for anaerobic digestion in an embodiment of the disclosed invention can comprise blood meal, green matter with high nitrogen content (e.g., clover or alfalfa), and waste matter.

Blood meal (“BM”) is a dry, inert powder produced by spray drying at low temperatures the fresh blood from animal processing plants. Whole blood is chilled and agitated to prevent coagulation and then centrifuged to remove foreign materials consisting of bone, hair, fat, and tissue. A final filtration or disintegration step is completed before the final drying process commences. The resulting dried product can be granulated or formed into pellets or pill. Blood meal (BM) has one of the highest non-synthetic sources of nitrogen available. Blood meal has excellent nutritional characteristics and contains >80% crude protein, with less than 1% total fat. Lower lipid concentrations decrease the VFA formation potential and thereby mitigate VFA inhibition when BM is used as a co-substrate. In contrast typical slaughterhouse wastes contain 15-30% total fat in both suspended and dissolved forms. BM has 14% available nitrogen from protein with crude fiber (carbohydrate content) below 1% by weight. BM is water soluble therefore essential micro nutrients are more readily bioavailable to the digester flora. Cellular absorption and assimilation of growth nutrients by digester flora is increased when compared to the digestion of suspended nutrients. Granular or pelletized blood meal can be easily shipped, transferred, and stored without the necessity for stringent health and safety procedures.

BM is very stable and can be stored under dry conditions for long periods with no decomposition or nutrient loss.

TABLE 1
Nutrient Profile of Blood Meal
AMINO ACID PROFILE

| | | | |
|---|---|---|---|
| Tryptophan | 1% | Alanine | 7.69% |
| Lysine | 7.0% | Valine | 5.2% |
| Histidine | 3.05% | Methionine** | 1% |
| Ammonia | 1.13% | Isoleucine | .8% |
| Arginine | 2.35% | Leucine | 10.3% |
| Aspartic Acid | 10.84% | Tyrosine | 2.34% |
| Threonine | 3.8% | Phenylalanine | 5.1% |
| Serine | 4.56% | Taurine | — |
| Glutamic Acid | 8.79% | Cystine | 1.4% |
| Glycine | 4.4% | Proline | 4.62% |

CRUDE PROTEIN: 86% (minimum)

### CRUDE FIBER: 1%

### NPK: 14-1-0.6

### SULFUR: 0.4%

### FAT: <1%

### ASH: 4.5%

### MOISTURE: 10%

COLOR: Dark Red to black

### Minerals & Vitamins

### CALCIUM: 0.28%

### PHOSPHORUS: 0.22%

### AVAILABLE PHOSPHORUS: 0.25%

### SALT EQUIVALENT: 0.65%

### SODIUM: 0.26%

### CHLORIDE: 0.39%

### POTASSIUM: 0.9%

### CHOLINE: 990 Mg/K

### DIGESTIBILITY: 94%

In contrast, slaughterhouse wastes (SHIN) are commonly available as aqueous slurries with high suspended solid concentrations. Digestion of suspended animal tissue, hair, and fats is enzymatically energy intensive and therefore growth nutrients are not as readily bioavailable when compared to BM. The mechanical separation and removal of animal tissues, fats, hair, and bone significantly decreases the sulfur content of BM. BM contains only 0.4% sulfur by weight verses 2-3% sulfur for slaughterhouse wastes. BM also contains lower concentrations of the sulfur containing amino acids, taurine, methionine, homocystine, and cysteine which further minimizes biosynthesis of hydrogen sulfide under anaerobic conditions, SHW are prone to decomposition with subsequent reduction of nutrient content during both storage and transportation. SHW must therefore be used immediately and cannot be stored statically. Odor control and vector attraction can be problematic when using raw animal wastes as co-substrates.

Sweet clover (“SC”) is a sweet-scented, upright, broad-leaved legume widely distributed over the world and has economic importance in the United States and Canada. Two common varieties of sweet clover found in the US and Canada are the white-flowered (Melilotus alba Dser) and the yellow-flowered (M. officinalis L). Yellow sweet clover is more drought tolerant, vigorous as a seedling, flowers earlier, and has spreading growth. Sweet clover has the greatest warm-weather biomass production of any legume (including alfalfa), tolerates alkaline soils, and can thus be used to reclaim saline areas. Sweet clover contains a high concentration of non-lignified fiber that improves the dewatering of digestate solids. Widely acclimated and self-reseeding, sweet clover can be seen growing on barren slopes, road ways, mining spoils, and in soils of low fertility. Sweet clover can tolerate a wide range of growth environments from sea level to 4,000 feet in altitude, including poor draining soils, heat, insect predation, plant diseases, and with as little as 6 inches of rain per year.

Sweet clover is a nutrient rich plant with 15% protein, approximately 2.5-4% nitrogen, with less than 1% total fat. Lower lipid concentrations found in SC decrease the VFA formation potential and thereby mitigate VFA inhibition when SC is used as a secondary co-substrate. The plant contains a high concentration of non-lignified fiber that improves the dewatering of digestate solids. The co-digestion of plant proteins (SC) with that of animal proteins (BM) balances the protein and amino acid profile as well as supplies additional raw plant nutrients to the digester blend, SC utilized as a digester co-substrate is the dried upper portion of the stalk, leaves, and flowers. The plant is mechanically harvested at ground level such that the roots are excluded from the harvest. Harvested plants are field dried and bailed for storage.

TABLE 2
Nutrient Profile of Sweet Clover

| Main analysis | Unit | Avg | SD | Min | Max |
|---|---|---|---|---|---|
| Dry matter | % as fed | 21.6 | | | |
| Crude protein | % DM | 15.3 | | | |
| Crude fibre | % DM | 29.4 | | | |
| Ether extract | % DM | 1.7 | | | |
| Ash | % DM | 13.1 | | | |
| Fat | % DM | 0.5 | | | * |
| Ruminant nutritive values | Unit | Avg | SD | Min | Max |
| OM digestibility, Ruminant | % | 65.8 | | | * |
| Energy digestibility, ruminants | % | 62.9 | | | * |
| DE ruminants | MJ/kg DM | 11.0 | | | * |
| ME ruminants | MJ/kg DM | 8.8 | | | * |
| Nitrogen digestibility, ruminants | % | 82.0 | | | |

The asterisk * indicates that the average value was obtained by an equation.

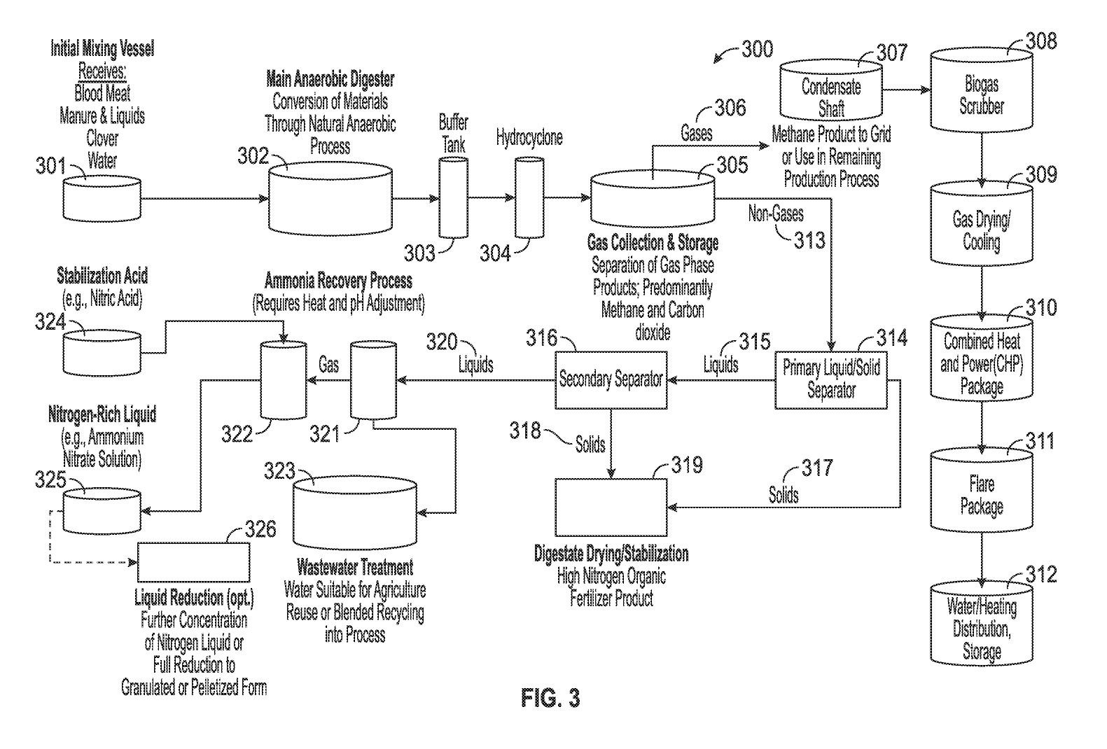

FIG. 3 illustrates an exemplary high level flow chart 300 of a method and system for high-nitrogen loading for ammonia processing via anaerobic digestion, in accordance with the disclosed embodiments. An initial mixing tank or vessel 101 can be utilized to receive and mix co-substrates comprising the blood meal, manure and liquids, sweet clover, and water. Organic Materials Research Institute (“OMRI”) certified organic blood meal can be transported via truck carrier and can be either blown or mechanically conveyed into conical storage silo. Auger flights transport the blood meal into two 250,000 gallon in-ground blending tanks each containing a process charge of dairy manure. The dosage of BM is controlled by weight monitoring differential pressure sensors. OMRI-certified organic sweet clover can be truck delivered and stored in covered storage-plate(s). Front end loaders are used to transport sweet clover to the blending tanks. Dosing control of sweet clover co-substrate is determined by volume to weight relationship of loader bucket. Hydration, maceration, and homogenization are accomplished in the initial mixing vessel 301 via gear reduced mixing props. It is understood that a plurality of initial mixing vessels 301 can be utilized in accordance with the disclosed embodiments.

The co-substrate components (e.g., blood meal, manure and liquids, sweet clover, and water) can be added to the main anaerobic digester 302 using solids tolerant sludge pumps during front end loading to augment and improve performance parameters for nitrogen, ammonia, and ammonium production. A waste stream or substrate having anaerobically biodegradable components can be fed into a main anaerobic digester 302 wherein the components react to biodegrade the components and produce biomass, ammonia, ammonium, and biogas. The organic matter is digested under anaerobic conditions in the digester using continuously stirred tank reactors while producing biogas and a final digested sludge with high nitrogen content. For example, effluent water with high nitrogen organic and/or inorganic supplements can be mixed to a 92% liquid and 8% solids mixture to load the main anaerobic digester 302. A “substrate” can include, for example, organic matter, such as animal material, plant material, animal feces, sewage sludge, industrial waste sludge, etc., and any water used to dilute such substrate.

Inside the main anaerobic digester 302, the co-substrate material is continuously mixed by a central, vertical agitator to convert the materials through the natural anaerobic process into biogas and solids. Microbes degrade the biological materials over an approximate 28-day period, for example, under a preferable mesophilic temperature range of 92 degrees to 104 degrees Fahrenheit. These standard biological materials used in anaerobic digestion are, however, relatively low in their nitrogen content. In the main anaerobic digester 302, the majority of the manure and high nitrogen organic and/or inorganic supplements are degraded and the volatile solids converted into biogas with a methane concentration of approximately 60%. Additionally, it is in this digestion process that the nitrogen in the manure and high nitrogen organic and/or inorganic supplements is converted to ammonia and ammonium forms.

The digester can be maintained under the following preferable conditions:

Digester type: Continuously stirred tank reactor, CSTR
Operational mode: Mesophilic digestion
Digester heating: Hot water
Gas collection: Pressure dome, flexible membrane
Digestion temperature: 98.8° F., 31.7° C.
Digester feed rate: 250,000 Gallons per day
HRT: 40-50 days equilibration, 28 days equilibrated
pH control: Biological regulated @ 7.4 units (no caustic addition)

Components can be added to the anaerobic digester 302 in the following exemplary quantities:

Blood meal: Qty 7.750 mt/yr (17.081.000 lb/yr); TS 6.975 mt/yr (TS 15.372.900 lb/yr); VS 5.580 mt/yr (VS 12.298.320 lb/yr); TN 1.116 mt/yr (TN 2.459.664 lb/yr); TP 91 mt/yr (TP 199.848 lb/yr); TK 49 mt/yr (TK 107.610 lb/yr).

Well Water: Qty 162.592 mt/yr (358.353.600 lb/yr); TS 292 mt/yr (TS 642.647 lb/yr); VS 0 mt/yr (VS 0 lb/yr); TN 0 mt/yr (TN 908 lb/yr); TP 0 mt/yr (TP 5 lb/yr); TK 0 mt/yr (TK 0 lb/yr).

Cow Manure and Flush Water: Qty 127.445 mt/yr (280.889.400 lb/yr); TS 19.754 mt/yr (TS 43.537.857 lb/yr); VS 14.124 mt/yr (VS 31.129.568 lb/yr); TN 626 mt/yr (TN 1.379.729 lb/yr); TP 113 mt/yr (TP 250.085 lb/yr); TK 299 mt/yr (TK 660.090 lb/yr).

Clover: Qty 182 mt/yr (401.500 lb/yr); TS 36 mt/yr (TS 80.300 lb/yr); VS 34 mt/yr (VS 75.482 lb/yr); TN 1 mt/yr (TN 2.088 lb/yr); TP 0 mt/yr (TP 562 lb/yr); TK 1 mt/yr (TK 2.489 lb/yr).

Equilibrated hydraulic retention time (HRT) within the digester is 28 days. After an exemplary 28-day digestion period, the degraded manure and high nitrogen organic and/or inorganic supplements pass through a buffer tank 303 and hydrocyclone 304. The gases 306 is then transferred to the gas collection and storage unit 305 while the non-gases 313 are transferred to the post digester tank for liquid/solid separation 314. A secondary or post digester and gas collector and storage unit 305 is provided for each of the primary digesters. Both the main anaerobic digester 302 and gas collector and storage unit 305 are equipped with mixer/agitators to ensure constant turn-over of tank contents. It is understood that a plurality of gas collection and storage units 305 can be utilized in accordance with the disclosed embodiments.

The gas collection and storage unit 305 can comprise a condensate shaft 307 to remove water from the methane, biogas scrubber 308 to remove hydrogen sulfide and CO₂ from the methane, methane gas drying/cooling 309 dried using a dedicated gas dryer with chiller, combined heat and power (CHP) package 310, flare package 311, and water heating/distribution/storage 312 to distribute hot and cold water throughout the digestion system. Conditioned methane gas is available for storage, transmission or to generate electrical power. Methane derived from the plant operation can also be used to fuel the digester boilers.

Exhausted post digester bottoms (digestate) can be purged after 28 days and pumped to dewatering presses. From the primary liquid/solid separator 314, the solids are transferred to digestate drying/stabilization 319 to produce a high nitrogen organic product. The liquids 315 can be transferred to a secondary separator 316 to separate additional solids 318. The solids 318 can then be transferred to the digestate drying/stabilization 319 to produce a high nitrogen organic product. From the secondary separator 316, the liquids 320 are then transferred to the ammonia recovery process 321, 322 that requires heat and pH adjustment. The liquid fraction then goes into a wastewater treatment process 323. The liquid fraction derived from ARP 321, 322 can be treated with a stabilizing acid 324 (e.g., nitric acid).

Following treatment, a nitrogen rich 325, high ammonia product is produced, in an optional step, a liquid reduction 326 is captured with a further concentration of nitrogen. Ammonia in the digestate liquid fraction will be in the form of ammonium bicarbonate. Stabilized ammonia concentrations are anticipated in the 4,000-4,400 ppm NH₄⁺-N range. The digestate liquid fraction stream can be directed to a commercial ammonia recovery system manufactured in the United States. The ammonia recovery system is a physical chemical process whereby ammonium salts are converted to gaseous ammonia by pH adjustment, purged from solution by temperature and pressure manipulations, then post treated using ammonia defusing membranes motivated by a specific liquid acid to form an ammonium salt (fertilizer). Alternatively, a full reduction to a high ammonia pelletized/granulated product is produced in the form of an ammonium salt, depending on the acid used for stabilization 324 and capture. Bio-solids are separated, dried, and pelletized using a commercial grain pelletizing system (not illustrated in FIG. 1). The use of OMRI certified co-substrates enhances the likelihood of OMRI certification for the pelletized solid.

### Co-Substrate Ratios:

The exemplary primary substrate, dairy manure, can be enriched with ratios of exemplary co-substrates blood meal and sweet clover for anaerobic digestion processes. Addition of blood meal and sweet clover can be determined by the chemical characteristics of the feed stock delivered to the digester. Ratios of blood meal and sweet clover are principally balanced against the nitrogen content of the primary digester substrate. When added in the correct ratios, blood meal and sweet clover can supply an enriched protein environment that effectively doubles the nitrogen content of the feedstock, increases methane production via increase acetate formation and introduces only minor concentrations of lipids.

The quantity of ammonia that will be generated from an anaerobic biodegradation of dairy manure using blood meal and sweet clover co-substrates can be estimated using the following stoichiometric relationship:

CₐH_{b}O_{c}N_{d}+((4a−b−2c+3d)/4)H₂O→((4a+b−2c−3d)/8)CH₄+((4a−b+2c+3d)/8)CO₂+dNH₃

Moles of protein (CₐH_{b}O_{c}N_{d}) are derived by proportioning the protein concentrations of the primary and co-substrates. Stoichiometry predicts ammonia concentrations will double as a result of doubling the protein load (1800-2000 ppm NH4-N digested dairy manure, 4000-4400 ppm protein enriched manure).

Elevated ammonia concentrations can be managed by digester acclimation favoring the proliferation of syntrophic acetate oxidizers (“SAO”) and hydrogen utilizing methanogens. The alternate SAO pathway to methane formation is activated by elevated levels of ammonia. Acetoclastic methanogens which account for 70-80% of methane produced are inhibited when ammonia concentrations exceed 1500 ppm (NH₄⁺—N/L). Methane production from acetate can still proceed via the SAO pathway even though the acetoclastic methanogens are inhibited. The development of the metabolic SAO pathway allows stable operation of mesophilic digestion processes with ammonia concentrations in the 4000-5000 ppm range. Generation time of an SAO culture was calculated to be approximately 28 days compared to 2-12 days for acetate utilizing methanogens. A longer hydraulic retention time (HRT) is therefore a preferable prerequisite to allow SAO to establish in the digester.

Table 3 illustrates the relative chemical composition of dairy manure from 11 dairies in the Central Valley area of California.

TABLE 3
Summary of properties of 29 solid manure samples;
(4 corral, 8 pond solids, 14 mechanical screen solids, 3 composts)

| Property | Unit | Median | Minimum | Maximum |
|---|---|---|---|---|
| Moisture Content | % wet wt. | 68 | 1 | 83 |
| Volatile solids | % dry wt. | 72 | 35 | 89 |
| Total Carbon | % dry wt. | 35.6 | 18.1 | 43.9 |
| Total N | % dry wt. | 2.1 | 1.2 | 3.5 |
| C:N | — | 16.1 | 9.3 | 33.4 |
| NH₄—N | Mg/kg dry wt. | 1346 | 13 | 6282 |
| NO₃—N | Mg/kg dry wt. | 9 | <1 | 312 |
| Total P | % dry wt. | 0.41 | 0.18 | 1.99 |
| Total K | % dry wt. | 0.57 | 0.15 | 4.37 |
| pH(sat'd paste) | — | 7.8 | 6.6 | 9.0 |
| EC(sat'd paste | mS/cm | 4.1 | 1.7 | 36 |
| extract) | | | | |

Co-substrate ratio of blood meal to sweet clover is maintained at approximately (40:1) on a weight to weight basis. Nitrogen content of the (40:1) blend is 13.7% W:W. The following dosing estimates are based on the digestion of 1,000,000 pounds of dairy manure with 68% water, 32% solids, and 1.2% W:W nominal nitrogen content. Primary substrate will be further diluted by 50% with make-up water to facilitate dosing into the anaerobic digester device. Nitrogen content of the primary substrate will be enriched by a factor of 1.8 with the addition of the two co-substrates.

Primary substrate: (1,000,000 lb)*(0.32 solid content)*(0.5 dilution) (0.012 N)=1,920 lb N

Nitrogen enrichment factor=1.8
Co-substrate BMSC blend (40:1)=13.7% N
Nitrogen enriched substrate=(10.8)*(1,920 lb N)=3,456 lb N target
Co-substrate BMSC dosage=(25,226 lb BMSC)/(1×10⁶ lb)

### Dosing Co-Substrates:

Commercial ammonia recovery systems are flexible processes that allow the use of different acids to produce different ammonium salt products. As an example, the use of hydrochloric acid yields ammonium chloride; sulfuric acid yields ammonium sulfate; nitric acid yields ammonium nitrate; and acetic acid yields ammonium acetate. Ammonium nitrate and citrate have high market values, therefore the subsequent marketing and sale of these products can create profit margin and accelerate return on investment. Maintaining high levels of ammonia in the digestate liquid phase is therefore highly desirable from a co-product manufacturing and economic perspective.

Several economic shortfalls for the exclusive digestion of dairy manure are the lower concentrations of ammonia and ammonium salts and reduced methane production. In most cases, the lower ammonia concentrations generated from the exclusive digestion of dairy manure does not economically allow for the capital expense of the ammonia recovery system. The use of high protein, low fat and low sulfur BMSC co-substrates stimulates ammonia and methane production, and minimizes sulfide, lipid, and VFA inhibitory responses. The treated bottoms from the ammonia recovery system are then delivered to a biological nitrification/de-nitrification waste treatment facility, then permit discharged. A 30-40% increase in methane production results with the aforementioned BMSC addition levels. Divergence of theoretical methane production (by calculation) as compared to preliminary pilot results are attributed to the variables stated above.

Stoichiometric models predict higher methane concentrations by increasing protein content of the feed stock. Co-digestion with BMSC at the proper concentrations allows an increase in both protein and nitrogen while limiting lipid content of the feed stock. Lipids and fats can generate more biogas per kilogram than that of protein; however, rising VFA concentrations can have major inhibitory effect on digestion primarily from the reduction of pH.

TABLE 4
Substrate Vs. Methane Production

| | Biogas | |
|---|---|---|
| Substrate | (nm3/kg)* | Methane (%) |
| Fat/Lipid (C57H104O6) | 1.4 | 70 |
| Protein (C5H7O2N) | 1.0 | 50 |
| Carbohydrate (C6H12O6) | 0.8 | 50 |

*At 1 atm, 0 C.

Stoichiometric models provide high estimates of methane production during anaerobic digestion but fail to take into consideration the following variables: digester design utilized, digester efficiency, biological efficiency of established digester flora, conversion of substrate, synthesis of new cellular material, kinetics of substrate degradation, influence of gas-liquid equilibrium on bacterial growth, influence of temperature on bacterial growth, and kinetics of product formation.

It will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following claims.

What is claimed is:

1. A method for high-nitrogen loading via anaerobic digestion, said method comprising:
feeding a substrate comprising a waste material having anaerobically biodegradable components into an anaerobic digester device, wherein said anaerobic digester device comprises a single stage anaerobic digester or a multi-stage anaerobic digester, wherein said anaerobic digester comprises monitoring temperature, mixing of organic waste, pH, and ammonia levels within a heated vessel of said anaerobic digester device;
feeding a co-substrate into said anaerobic digester device with said substrate; and
co-digesting said substrate and said co-substrate in said anaerobic digester device to produce a biomass, a biogas, and a waste product, wherein nitrogen in said waste product and a high nitrogen organic and/or inorganic supplements are converted to a material comprising ammonia.

2. The method of claim 1 wherein:
said co-substrate comprises at least one of blood meal and sweet clover, wherein said co-substrate co-digests with said substrate and provides nitrogen and nutrients missing from digestion that prevent inhibiting substances from affecting methanogenesis, wherein co-digestion of said at least one of blood meal and sweet clover balances a protein and amino acid profile within said digester and supplies additional raw plant nutrients to said digester; and
wherein said waste material comprises at least one of manure, dairy manure, and other manure based wastes.

3. The method of claim 1 further comprising supplementing said heated vessel with blood meal and sweet clover and controlling said supplements via a pumping system.

4. The method of claim 1 further comprising:
recovering said ammonia in an ammonia recovery process from a liquid fraction; and
stabilizing said ammonia and capturing said ammonia using an acid resulting in a high concentration ammonia liquid product or high ammonium salt, said salt comprising a pelletized or granulated product depending on said acid used for said stabilization and said capture.

5. The method of claim 1 wherein said heated vessel receives organic waste, blood meal, and clover, wherein said organic waste is retained in said anaerobic digester for 20 to 40 days, and is fed continuously or in batches, and wherein fresh organic material is mixed with partially-digested material in said heated vessel.

6. The method of claim 1 further comprising overcoming inhibitory materials produced during digestion by introducing blood meal and sweet clover to said digester to increase protein load within said digester and yielding an increased amount of methane, biomass, and low toxicity ammonium.

7. The method of claim 1 wherein said anaerobic digester comprises a single stage digester, said signal stage digester comprising a vessel with an attached manure reception pit with an attached pump for pumping influent into said vessel, wherein blood meal and clover additives enter said vessel through said pump in said manure reception pit, wherein mixers mix influent that enters said vessel with said blood meal and clover.

8. The method of claim 1 wherein said anaerobic digester comprises a multi-stage digester, said multi-stage digester comprising a first vessel and a second vessel, wherein said first vessel has an attached manure reception pit with an attached pump for pumping influent into said vessel, wherein blood meal and clover additives enter said first vessel through said pump in said manure reception pit, wherein mixers mix influent that enters said first vessel with said blood meal and clover, wherein hydrolysis occurs in said first vessel and methanogenesis occurs in said second vessel, wherein mixed sludge from said first vessel is pumped into said second vessel, wherein supernatant is separated from digested sludge and scum in said second vessel.

9. The method of claim 1 further comprising calculating a quantity of generated ammonia from an anaerobic biodegradation of dairy manure using blood meal and sweet clover co-substrates via an equation CₐH_{b}O_{c}N_{d}+((4a−b−2c+3d)/4)H₂O→((4a+b−2c−3d)/8)CH₄+(4a−b+2c+3d)/8)CO₂+dNH₃, wherein moles of protein (CₐH_{b}O_{c}N_{d}) are derived by proportioning protein concentrations of said substrate and said blood meal and sweet clover co-substrates.

10. A system for high-nitrogen loading via anaerobic digestion, said system comprising:
a substrate comprising a waste material having anaerobically biodegradable components fed into an anaerobic digester device, wherein said anaerobic digester device comprises a single stage anaerobic digester or a multi stage anaerobic digester, wherein said anaerobic digester comprises monitoring temperature, mixing of organic waste, pH, and ammonia levels within a heated vessel of said anaerobic digester device;
a co-substrate fed into said anaerobic digester device with said substrate; and
said substrate and said co-substrate co-digested in said anaerobic digester device to produce a biomass, a biogas, and a waste product, wherein nitrogen in said waste product and a high nitrogen organic and/or inorganic supplements are converted to a material comprising ammonia.

11. The system of claim 10 wherein:
said co-substrate comprises at least one of blood meal and sweet clover, wherein said co-substrate co-digests with said substrate and provides nitrogen and nutrients missing from digestion that prevent inhibiting substances from affecting methanogenesis, wherein co-digestion of said at least one of blood meal and sweet clover balances a protein and amino acid profile within said anaerobic digester device and supplies additional raw plant nutrients to said anaerobic digester device, wherein said co-substrate supplements said substrate within said heated vessel as controlled by a pumping system; and
wherein said waste material comprises at least one of manure, dairy manure, and other manure based wastes.

12. The method of claim 10 further comprising:
recovering said ammonia in an ammonia recovery process from a liquid fraction; and
stabilizing said ammonia and capturing said ammonia using an acid resulting in a high concentration ammonia liquid product or high ammonium salt, said salt comprising a pelletized or granulated product depending on said acid used for said stabilization and said capture.

13. The system of claim 10 wherein said heated vessel receives organic waste, blood meal, and clover, wherein said organic waste is retained in said anaerobic digester for 20 to 40 days, and is fed continuously or in batches, and wherein fresh organic material is mixed with partially-digested material in said heated vessel.

14. The system of claim 10 further comprising overcoming inhibitory materials produced during digestion by introducing blood meal and sweet clover to said digester to increase protein load within said digester and yielding an increased amount of methane, biomass, and low toxicity ammonium.

15. The system of claim 10 wherein said anaerobic digester comprises a single stage digester, said signal stage digester comprising a vessel with an attached manure reception pit with an attached pump for pumping influent into said vessel, wherein blood meal and clover additives enter said vessel through said pump in said manure reception pit, wherein mixers mix influent that enters said vessel with said blood meal and clover.

16. The system of claim 10 wherein said anaerobic digester comprises a multi-stage digester, said multi-stage digester comprising a first vessel and a second vessel, wherein said first vessel has an attached manure reception pit with an attached pump for pumping influent into said vessel, wherein blood meal and clover additives enter said first vessel through said pump in said manure reception pit, wherein mixers mix influent that enters said first vessel with said blood meal and clover, wherein hydrolysis occurs in said first vessel and methanogenesis occurs in said second vessel, wherein mixed sludge from said first vessel is pumped into said second vessel, wherein supernatant is separated from digested sludge and scum in said second vessel.

17. An apparatus for high-nitrogen loading via anaerobic digestion, said apparatus comprising:
an anaerobic digester device and a substrate comprising a waste material having anaerobically biodegradable components fed into said anaerobic digester device, wherein said anaerobic digester device comprises a single stage anaerobic digester or a multi-stage anaerobic digester, wherein said anaerobic digester comprises monitoring temperature, mixing of organic waste, pH, and ammonia levels within a heated vessel of said anaerobic digester device;
a co-substrate fed into said anaerobic digester device with said substrate; and
said substrate and said co-substrate co-digested in said anaerobic digester device to produce a biomass, a biogas, and a waste product, wherein nitrogen in said waste product and a high nitrogen organic and/or inorganic supplements are converted to a material comprising ammonia.

18. The apparatus of claim 17 wherein said anaerobic digester comprises:
a single stage digester, said signal stage digester comprising a vessel with an attached manure reception pit with an attached pump for pumping influent into said vessel, wherein blood meal and clover additives enter said vessel through said pump in said manure reception pit, wherein mixers mix influent that enters said vessel with said blood meal and clover; or
a multi-stage digester, said multi-stage digester comprising a first vessel and a second vessel, wherein said first vessel has an attached manure reception pit with an attached pump for pumping influent into said vessel, wherein blood meal and clover additives enter said first vessel through said pump in said manure reception pit, wherein mixers mix influent that enters said first vessel with said blood meal and clover, wherein hydrolysis occurs in said first vessel and methanogenesis occurs in said second vessel, wherein mixed sludge from said first vessel is pumped into said second vessel, wherein supernatant is separated from digested sludge and scum in said second vessel.

19. The apparatus of claim 17 wherein:
said co-substrate comprises at least one of blood meal and sweet clover, wherein said co-substrate co-digests with said substrate and provides nitrogen and nutrients missing from digestion that prevent inhibiting substances from affecting methanogenesis, wherein co-digestion of said at least one of blood meal and sweet clover balances a protein and amino acid profile within said anaerobic digester device and supplies additional raw plant nutrients to said anaerobic digester device, wherein said co-substrate supplements said substrate within said heated vessel as controlled by a pumping system; and
wherein said waste material comprises at least one of manure, dairy manure, and other manure based wastes.

20. The apparatus of claim 17 wherein said heated vessel receives organic waste, blood meal, and clover, wherein said organic waste is retained in said anaerobic digester for 20 to 40 days, and is fed continuously or in batches, and wherein fresh organic material is mixed with partially-digested material in said heated vessel.
